(19) United States
(12) Patent Application Publication
(10) Pub. No.: US 2025/0331918 A1
(43) Pub. Date: Oct. 30, 2025

(54) PRETREATMENT WAVEFORM FOR IRREVERSIBLE ELECTROPORATION

(71) Applicant: Boston Scientific Scimed, Inc., Maple Grove, MN (US)

(72) Inventor: Allan Charles SHUROS, St. Paul, MN (US)

(21) Appl. No.: 19/260,037

(22) Filed: Jul. 3, 2025

Related U.S. Application Data
(63) Continuation of application No. 17/488,217, filed on Sep. 28, 2021, now Pat. No. 12,349,964.
(60) Provisional application No. 63/085,452, filed on Sep. 30, 2020.

(51) Int. Cl. A61B 18/14; A61B 18/00; A61B 18/12

(52) U.S. Cl. CPC A61B 18/1492; A61B 18/1206; A61B 2018/00577; A61B 2018/00613; A61B 2018/1253; A61B 2018/126; A61B 2562/0219

(57) ABSTRACT

An electroporation ablation system for treating targeted tissue in a patient. The electroporation ablation system including an ablation catheter and an electroporation generator. The ablation catheter including a handle, a shaft having a distal end, and catheter electrodes situated at the distal end of the shaft and spatially arranged to generate electric fields in the targeted tissue in response to electrical pulses. The electroporation generator operatively coupled to the catheter electrodes and configured to deliver the electrical pulses in an irreversible electroporation pulse sequence that includes a preconditioning pulse sequence and an electroporation pulse sequence to one or more catheter electrodes. Wherein the preconditioning pulse sequence includes preconditioning electrical pulses configured to cause electrolysis near the targeted tissue and tetanizing skeletal muscle stimulation in the patient.

### CROSS REFERENCE TO RELATED APPLICATION

This application is a continuation of and claims the benefit of U.S. patent application Ser. No. 17/488,217, filed Sep. 28, 2021, which claims the benefit of Provisional Application No. 63/085,452, filed Sep. 30, 2020, each of which is herein incorporated by reference in its entirety.

### TECHNICAL FIELD

The present disclosure relates to medical apparatus, systems, and methods for ablating tissue in a patient. More specifically, the present disclosure relates to medical apparatus, systems, and methods for ablation of tissue by electroporation.

### BACKGROUND

Ablation procedures are used to treat many different conditions in patients. Ablation may be used to treat cardiac arrhythmias, benign tumors, cancerous tumors, and to control bleeding during surgery. Usually, ablation is accomplished through thermal ablation techniques including radio-frequency (RF) ablation and cryoablation. In RF ablation, a probe is inserted into the patient and radio frequency waves are transmitted through the probe to the surrounding tissue. The radio frequency waves generate heat, which destroys surrounding tissue and cauterizes blood vessels. In cryoablation, a hollow needle or cryoprobe is inserted into the patient and cold, thermally conductive fluid is circulated through the probe to freeze and kill the surrounding tissue. RF ablation and cryoablation techniques indiscriminately kill tissue through cell necrosis, which may damage or kill otherwise healthy tissue, such as tissue in the esophagus, phrenic nerve cells, and tissue in the coronary arteries.

Another ablation technique uses electroporation. In electroporation, or electro-permeabilization, an electric field is applied to cells to increase the permeability of the cell membrane. The electroporation may be reversible or irreversible, depending on the strength of the electric field. If the electroporation is reversible, the increased permeability of the cell membrane may be used to introduce chemicals, drugs, and/or deoxyribonucleic acid (DNA) into the cell, prior to the cell healing and recovering. If the electroporation is irreversible, the affected cells are killed through apoptosis.

Irreversible electroporation (IRE) may be used as a nonthermal ablation technique. In IRE, trains of short, high voltage pulses are used to generate electric fields that are strong enough to kill cells through apoptosis. In ablation of cardiac tissue, IRE may be a safe and effective alternative to the indiscriminate killing of thermal ablation techniques, such as RF ablation and cryoablation. IRE may be used to kill targeted tissue, such as myocardium tissue, by using an electric field strength and duration that kills the targeted tissue but does not permanently damage other cells or tissue, such as non-targeted myocardium tissue, red blood cells, vascular smooth muscle tissue, endothelium tissue, and nerve cells.

In some IRE procedures, the electroporation electrical pulses cause the unwanted side effect of skeletal muscle stimulation (SMS) and engagement. One way to reduce SMS, is to refine the IRE electrical pulses, such that the pulses are optimized to avoid SMS. Often this results in having a smaller ablation electric field and in creating smaller lesions. A way of delivering effective IRE energies while avoiding SMS is needed.

### SUMMARY

In Example 1, an electroporation ablation system for treating targeted tissue in a patient. The electroporation ablation system including an ablation catheter and an electroporation generator. The ablation catheter including a handle, a shaft having a distal end, and catheter electrodes situated at the distal end of the shaft and spatially arranged to generate electric fields in the targeted tissue in response to electrical pulses. The electroporation generator operatively coupled to the catheter electrodes and configured to deliver the electrical pulses in an irreversible electroporation pulse sequence that includes a preconditioning pulse sequence and an electroporation pulse sequence to one or more catheter electrodes. Wherein the preconditioning pulse sequence includes preconditioning electrical pulses configured to cause electrolysis near the targeted tissue and tetanizing skeletal muscle stimulation in the patient.

In Example 2, the electroporation ablation system of Example 1, comprising a surface patch electrode attached to the patient and configured to generate electric fields in the patient in response to the electrical pulses.

In Example 3, the electroporation ablation system of Example 2, wherein the preconditioning pulse sequence includes unipolar electrical pulses that are sourced from the surface patch electrode and sunk through the one or more catheter electrodes.

In Example 4, the electroporation ablation system of Example 2, wherein the preconditioning pulse sequence includes unipolar electrical pulses that are sourced from the one or more catheter electrodes and sunk through the surface patch electrode.

In Example 5, the electroporation ablation system of any of Examples 1-4, wherein the preconditioning pulse sequence includes bipolar electrical pulses that are sourced from at least one of the one or more catheter electrodes and sunk through at least another one of the one or more catheter electrodes.

In Example 6, the electroporation ablation system of any of Examples 1-5, wherein the preconditioning pulse sequence includes preconditioning pulses delivered at a selected frequency.

In Example 7, the electroporation ablation system of any of Examples 1-6, wherein the preconditioning pulse sequence includes preconditioning pulses ramped up in voltage from a lower voltage to a higher voltage over time.

In Example 8, the electroporation ablation system of any of Examples 1-7, wherein the preconditioning pulse sequence includes preconditioning pulses that include an exponentially decaying backside waveform that causes electrolysis near the targeted tissue.

In Example 9, the electroporation ablation system of any of Examples 1-8, wherein the preconditioning pulse sequence includes preconditioning pulses that are monophasic.

In Example 10, the electroporation ablation system of any of Examples 1-9, wherein the irreversible electroporation pulse sequence, including the preconditioning pulse sequence and the electroporation pulse sequence, is delivered to the patient in one or more of a refractory time of a heart of the patient, less than 330 milliseconds, and in a 100-250 millisecond window.

In Example 11, the electroporation ablation system of any of Examples 1-10, wherein the electroporation pulse sequence is delivered within the preconditioning pulse sequence.

In Example 12, the electroporation ablation system of any of Examples 1-11, wherein the electroporation pulse sequence includes bipolar electrical pulses delivered to one or more catheter electrode pairs of the catheter electrodes.

In Example 13, the electroporation ablation system of any of Examples 1-12, comprising an accelerometer configured to monitor skeletal muscle stimulation of the patient and wherein the electroporation ablation system is a closed loop system such that the electroporation generator is configured to deliver the preconditioning pulse sequence, detect tetany in the patient, and then deliver the electroporation pulse sequence, and wherein local impedance is measured to calculate pre-ablation and post-ablation values to evaluate lesion efficacy.

In Example 14, an electroporation ablation system for treating targeted tissue in a patient. The electroporation ablation system including an ablation catheter and an electroporation generator. The ablation catheter including a handle, a shaft having a distal end, and catheter electrodes situated at the distal end of the shaft and spatially arranged to generate electric fields in the targeted tissue in response to electrical pulses. The electroporation generator operatively coupled to multiple electrodes including one or more of a surface patch electrode and one or more catheter electrodes and configured to deliver the electrical pulses in an irreversible electroporation pulse sequence that includes a preconditioning pulse sequence and an electroporation pulse sequence to the multiple electrodes, wherein the electroporation generator delivers the electroporation pulse sequence during the preconditioning pulse sequence.

In Example 15, the electroporation ablation system of Example 14, wherein the preconditioning pulse sequence includes electrical pulses configured to cause electrolysis near the targeted tissue and tetanizing skeletal muscle stimulation in the patient.

In Example 16, an electroporation ablation system for treating targeted tissue in a patient. The electroporation ablation system including an ablation catheter and an electroporation generator. The ablation catheter including a handle, a shaft having a distal end, and catheter electrodes situated at the distal end of the shaft and spatially arranged to generate electric fields in the targeted tissue in response to electrical pulses. The electroporation generator operatively coupled to the catheter electrodes and configured to deliver the electrical pulses in an irreversible electroporation pulse sequence that includes a preconditioning pulse sequence and an electroporation pulse sequence to one or more catheter electrodes, wherein the preconditioning pulse sequence includes preconditioning electrical pulses configured to cause electrolysis near the targeted tissue and tetanizing skeletal muscle stimulation in the patient.

In Example 17, the electroporation ablation system of Example 16, comprising a surface patch electrode attached to the patient and configured to generate electric fields in the patient in response to the electrical pulses.

In Example 18, the electroporation ablation system of Example 16, wherein the preconditioning pulse sequence includes unipolar electrical pulses that are sourced from the surface patch electrode and sunk through the one or more catheter electrodes.

In Example 19, the electroporation ablation system of Example 16, wherein the preconditioning pulse sequence includes unipolar electrical pulses that are sourced from the one or more catheter electrodes and sunk through the surface patch electrode.

In Example 20, the electroporation ablation system of Example 16, wherein the preconditioning pulse sequence includes bipolar electrical pulses that are sourced from at least one of the one or more catheter electrodes and sunk through at least another one of the one or more catheter electrodes.

In Example 21, the electroporation ablation system of Example 16, wherein the preconditioning pulse sequence includes preconditioning pulses delivered at a selected frequency.

In Example 22, the electroporation ablation system of Example 16, wherein the preconditioning pulse sequence includes preconditioning pulses ramped up in voltage from a lower voltage to a higher voltage over time.

In Example 23, the electroporation ablation system of Example 16, wherein the preconditioning pulse sequence includes preconditioning pulses that include an exponentially decaying backside waveform that causes electrolysis near the targeted tissue.

In Example 24, the electroporation ablation system of Example 16, wherein the preconditioning pulse sequence includes preconditioning pulses that are monophasic.

In Example 25, the electroporation ablation system of Example 16, wherein the irreversible electroporation pulse sequence, including the preconditioning pulse sequence and the electroporation pulse sequence, is delivered to the patient in one or more of a refractory time of a heart of the patient, less than 330 milliseconds, and in a 100-250 millisecond window.

In Example 26, the electroporation ablation system of Example 16, wherein the electroporation pulse sequence is delivered within the preconditioning pulse sequence.

In Example 27, the electroporation ablation system of Example 16, wherein the electroporation pulse sequence includes bipolar electrical pulses delivered to one or more catheter electrode pairs of the catheter electrodes.

In Example 28, the electroporation ablation system of Example 16, comprising an accelerometer configured to monitor skeletal muscle stimulation of the patient and wherein the electroporation ablation system is a closed loop system such that the electroporation generator is configured to deliver the preconditioning pulse sequence, detect tetany in the patient, and then deliver the electroporation pulse sequence, and wherein local impedance is measured to calculate pre-ablation and post-ablation values to evaluate lesion efficacy.

In Example 29, an electroporation ablation system for treating targeted tissue in a patient. The electroporation ablation system including an ablation catheter and an electroporation generator. The ablation catheter including a handle, a shaft having a distal end, and catheter electrodes situated at the distal end of the shaft and spatially arranged to generate electric fields in the targeted tissue in response to electrical pulses. The electroporation generator operatively coupled to multiple electrodes including one or more of a surface patch electrode and one or more catheter electrodes and configured to deliver the electrical pulses in an irreversible electroporation pulse sequence that includes a preconditioning pulse sequence and an electroporation pulse sequence to the multiple electrodes, wherein the electroporation generator delivers the electroporation pulse sequence during the preconditioning pulse sequence.

In Example 30, the electroporation ablation system of Example 29, wherein the preconditioning pulse sequence includes preconditioning electrical pulses configured to cause electrolysis near the targeted tissue and tetanizing skeletal muscle stimulation in the patient.

In Example 31, the electroporation ablation system of Example 29, wherein the electroporation pulse sequence includes bipolar electrical pulses delivered to selected pairs of the catheter electrodes.

In Example 32, a method of ablating targeted tissue in a patient by irreversible electroporation. The method comprising delivering an irreversible electroporation pulse sequence including delivering a preconditioning pulse sequence between multiple electrodes including one or more of a surface patch electrode and one or more catheter electrodes on a catheter to cause electrolysis near the targeted tissue and tetanizing skeletal muscle stimulation in the patient, and delivering an electroporation pulse sequence to the multiple electrodes to cause irreversible electroporation ablation of the targeted tissue.

In Example 33, the method of Example 32, wherein delivering a preconditioning pulse sequence includes delivering electrical pulses that ramp up in voltage from a lower voltage to a higher voltage over time and wherein one or more of the electrical pulses include an exponentially decaying backside waveform.

In Example 34, the method of Example 32, wherein the electroporation pulse sequence is delivered during the preconditioning pulse sequence.

In Example 35, the method of Example 32, comprising monitoring an accelerometer on the patient and in a closed loop system, delivering the preconditioning pulse sequence to achieve tetany in the patient, detecting tetany in the patient via the accelerometer, and delivering the electroporation pulse sequence after tetany has been achieved.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 is a diagram illustrating an exemplary clinical setting for treating a patient and for treating a heart of the patient, using an electrophysiology system, in accordance with embodiments of the subject matter of the disclosure.

FIG. 2A is a diagram illustrating a distal end of a shaft included in a catheter and interactions between electrode pairs, in accordance with embodiments of the subject matter of the disclosure.

FIG. 2B is a diagram illustrating axial electric fields generated by interactions between electrode pairs, in accordance with embodiments of the subject matter of the disclosure.

FIG. 2C is a diagram illustrating circumferential electric fields generated by interactions between electrode pairs in the catheter, in accordance with embodiments of the subject matter of the disclosure.

FIG. 3 is a diagram illustrating an IRE pulse sequence that includes a preconditioning pulse sequence and an electroporation pulse sequence, in accordance with embodiments of the subject matter of the disclosure.

FIG. 4 is a flow chart diagram illustrating a method of ablating targeted tissue in a patient by irreversible electroporation, in accordance with embodiments of the subject matter of the disclosure.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

The following detailed description is exemplary in nature and is not intended to limit the scope, applicability, or configuration of the invention in any way. Rather, the following description provides some practical illustrations for implementing exemplary embodiments of the present invention. Examples of constructions, materials, and/or dimensions are provided for selected elements. Those skilled in the art will recognize that many of the noted examples have a variety of suitable alternatives.

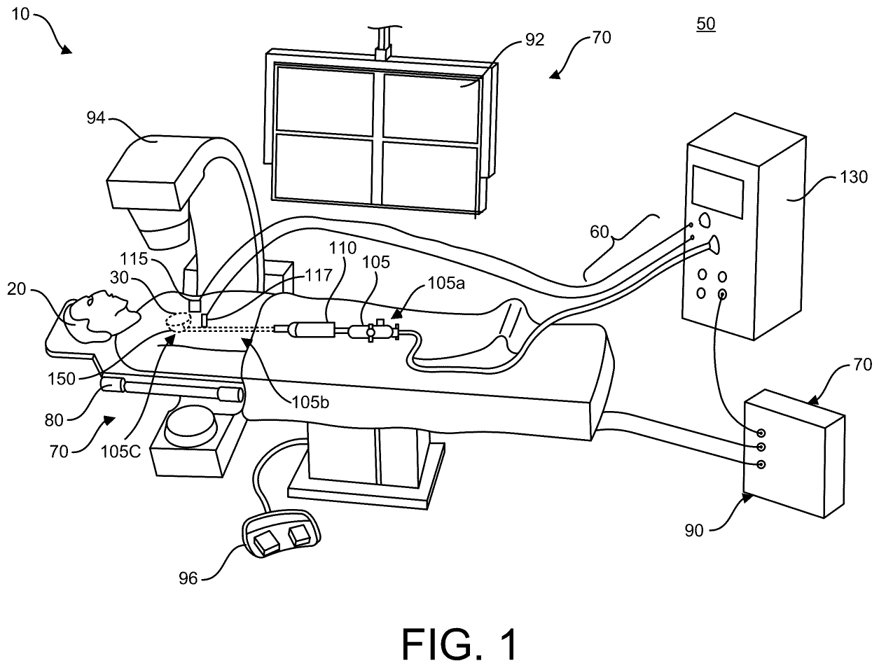

FIG. 1 is a diagram illustrating an exemplary clinical setting 10 for treating a patient 20, and for treating a heart 30 of the patient 20, using an electrophysiology system 50, in accordance with embodiments of the subject matter of the disclosure. The electrophysiology system 50 includes an electroporation system 60 and an electro-anatomical mapping (EAM) system 70, which includes a localization field generator 80, a mapping and navigation controller 90, and a display 92. Also, the clinical setting 10 includes additional equipment such as imaging equipment 94 (represented by the C-arm) and various controller elements, such as a foot controller 96, configured to allow an operator to control various aspects of the electrophysiology system 50. As will be appreciated by the skilled artisan, the clinical setting 10 may have other components and arrangements of components that are not shown in FIG. 1.

The electroporation system 60 includes an electroporation catheter 105, an introducer sheath 110, a surface patch electrode 115, and an electroporation generator 130. Also, in embodiments, the electroporation system 60 includes an accelerometer 117, where the accelerometer 117 can be a separate sensor or part of the surface electrode patch 115. Additionally, the electroporation system 60 includes various connecting elements (e.g., cables, umbilicals, and the like) that operate to functionally connect the components of the electroporation system 60 to one another and to the components of the EAM system 70. This arrangement of connecting elements is not of critical importance to the present disclosure, and one skilled in the art will recognize that the various components described herein may be interconnected in a variety of ways.

In embodiments, the electroporation system 60 is configured to deliver electric field energy to targeted tissue in the patient's heart 30 to create tissue apoptosis, rendering the tissue incapable of conducting electrical signals. The electroporation generator 130 is configured to control functional aspects of the electroporation system 60. In embodiments, the electroporation generator 130 is operable as a pulse generator for generating and supplying pulse sequences to the electroporation catheter 105 and the surface patch electrode 115, as described in greater detail herein. In embodiments, the electroporation generator 130 is operable to receive sensed signals from the accelerometer 117 and based on the received sensed signals act as a pulse generator for generating and supplying pulse sequences to the electroporation catheter 105 and the surface patch electrode 115, as described in greater detail herein.

In embodiments, the electroporation generator 130 includes one or more controllers, microprocessors, and/or computers that execute code out of memory to control and/or perform the functional aspects of the electroporation catheter system 60. In embodiments, the memory may be part of the one or more controllers, microprocessors, and/or computers, and/or part of memory capacity accessible through a network, such as the world wide web.

In embodiments, the introducer sheath 110 is operable to provide a delivery conduit through which the electroporation catheter 105 may be deployed to the specific target sites within the patient's heart 30. It will be appreciated, however, that the introducer sheath 110 is illustrated and described herein to provide context to the overall electrophysiology system 50, but it is not critical to the novel aspects of the various embodiments described herein.

The EAM system 70 is operable to track the location of the various functional components of the electroporation system 60, and to generate high-fidelity three-dimensional anatomical and electro-anatomical maps of the cardiac chambers of interest. In embodiments, the EAM system 70 may be the RHYTHMIA™ HDx mapping system marketed by Boston Scientific Corporation. Also, in embodiments, the mapping and navigation controller 90 of the EAM system 70 includes one or more controllers, microprocessors, and/or computers that execute code out of memory to control and/or perform functional aspects of the EAM system 70, where the memory, in embodiments, may be part of the one or more controllers, microprocessors, and/or computers, and/or part of memory capacity accessible through a network, such as the world wide web.

As will be appreciated by the skilled artisan, the depiction of the electrophysiology system 50 shown in FIG. 1 is intended to provide a general overview of the various components of the system 50 and is not in any way intended to imply that the disclosure is limited to any set of components or arrangement of the components. For example, the skilled artisan will readily recognize that additional hardware components, e.g., breakout boxes, workstations, and the like, may and likely will be included in the electrophysiology system 50.

The EAM system 70 generates a localization field, via the field generator 80, to define a localization volume about the heart 30, and one or more location sensors or sensing elements on the tracked device(s), e.g., the electroporation catheter 105, generate an output that may be processed by the mapping and navigation controller 90 to track the location of the sensor, and consequently, the corresponding device, within the localization volume. In the illustrated embodiment, the device tracking is accomplished using magnetic tracking techniques, whereby the field generator 80 is a magnetic field generator that generates a magnetic field defining the localization volume, and the location sensors on the tracked devices are magnetic field sensors.

In other embodiments, impedance tracking methodologies may be employed to track the locations of the various devices. In such embodiments, the localization field is an electric field generated, for example, by an external field generator arrangement, e.g., surface electrodes, by intra-body or intra-cardiac devices, e.g., an intracardiac catheter, or both. In these embodiments, the location sensing elements may constitute electrodes on the tracked devices that generate outputs received and processed by the mapping and navigation controller 90 to track the location of the various location sensing electrodes within the localization volume.

In embodiments, the EAM system 70 is equipped for both magnetic and impedance tracking capabilities. In such embodiments, impedance tracking accuracy can, in some instances, be enhanced by first creating a map of the electric field induced by the electric field generator within the cardiac chamber of interest using a probe equipped with a magnetic location sensor, as is possible using the aforementioned RHYTHMIA HDx™ mapping system. One exemplary probe is the INTELLAMAP ORION™ mapping catheter marketed by Boston Scientific Corporation.

Regardless of the tracking methodology employed, the EAM system 70 utilizes the location information for the various tracked devices, along with cardiac electrical activity acquired by, for example, the electroporation catheter 105 or another catheter or probe equipped with sensing electrodes, to generate, and display via the display 92, detailed three-dimensional geometric anatomical maps or representations of the cardiac chambers as well as electro-anatomical maps in which cardiac electrical activity of interest is superimposed on the geometric anatomical maps. Furthermore, the EAM system 70 may generate a graphical representation of the various tracked devices within the geometric anatomical map and/or the electro-anatomical map.

While the EAM system 70 is shown in combination with the electroporation system 60 to provide a comprehensive depiction of an exemplary clinical setting 10, the EAM system 70 is not critical to the operation and functionality of the electroporation system 60. That is, in embodiments, the electroporation system 60 can be employed independently of the EAM system 70 or any comparable electro-anatomical mapping system.

In the illustrated embodiment, the electroporation catheter 105 includes a handle 105a, a shaft 105b, and an electroporation electrode arrangement 150, which is described further hereinafter. The handle 105a is configured to be operated by a user to position the electroporation electrode arrangement 150 at the desired anatomical location. The shaft 105b has a distal end 105c and generally defines a longitudinal axis of the electroporation catheter 105. As shown, the electroporation electrode arrangement 150 is located at or proximate the distal end 105c of the shaft 105b. In embodiments, the electroporation electrode arrangement 150 is electrically coupled to the electroporation generator 130, to receive electrical pulse sequences or pulse trains, thereby selectively generating electrical fields for ablating the target tissue by irreversible electroporation.

In embodiments, the surface patch electrode 115 includes a conductive electrode that can be attached to the body of the patient 20, such as to the thorax of the patient. The surface patch electrode 115, including the conductive electrode, is electrically coupled to the electroporation generator 130 to act as a return path or sink for electrical energy in the system and to receive electrical pulse sequences or pulse trains from the electroporation generator 130, thereby acting as a source for electrical energy and selectively generating electrical fields for ablating the target tissue by irreversible electroporation. In embodiments, the surface patch electrode 115 acts as a return or sink for electrical energy received by the electroporation catheter 105 and the electroporation electrode arrangement 150. In embodiments, the surface patch electrode 115 acts as a source for electrical energy and the electroporation catheter 105 including the electroporation electrode arrangement 150 acts as the return or sink for the sourced electrical energy.

The electroporation system 60 is operable to generate an IRE pulse sequence that includes a preconditioning (pretreatment) pulse sequence and an electroporation pulse sequence. The IRE pulse sequence is configured to ablate targeted tissue. In embodiments, the preconditioning pulse sequence is a series of electrical pulses that ramp up in magnitude to tetanize skeletal muscle tissue and to provide electrolysis near targeted tissue. In embodiments, the electroporation pulse sequence is a series of electroporation pulses configured to cause irreversible damage to the targeted tissue.

In embodiments, the electroporation system 60 includes the accelerometer 117 that may be attached to the body of the patient 20, such as to the thorax of the patient, and electrically coupled to the electroporation generator 130. The accelerometer 117 is configured to sense contraction of the skeletal muscle system of the patient to detect tetany. The signals from the accelerometer 117 are received by the electroporation generator 130, which processes the signals to determine whether the skeletal muscle system of the patient is contracting and whether tetany has been achieved. In embodiments, the electroporation generator 130 is configured to provide the electroporation pulse sequence only after tetany has been achieved in the patient.

In embodiments, the electroporation system 60 acts as a closed system with the surface accelerometer 117 monitoring chest vibrations and the electroporation generator 130 modulating pulses until tetany is achieved and then the electroporation generator 130 delivers the electroporation pulses. Also, in embodiments, the local impedance of the target tissue and tissue surrounding the target tissue can be measured during this time to calculate pre-ablation and post-ablation values for evaluation of the lesion efficacy.

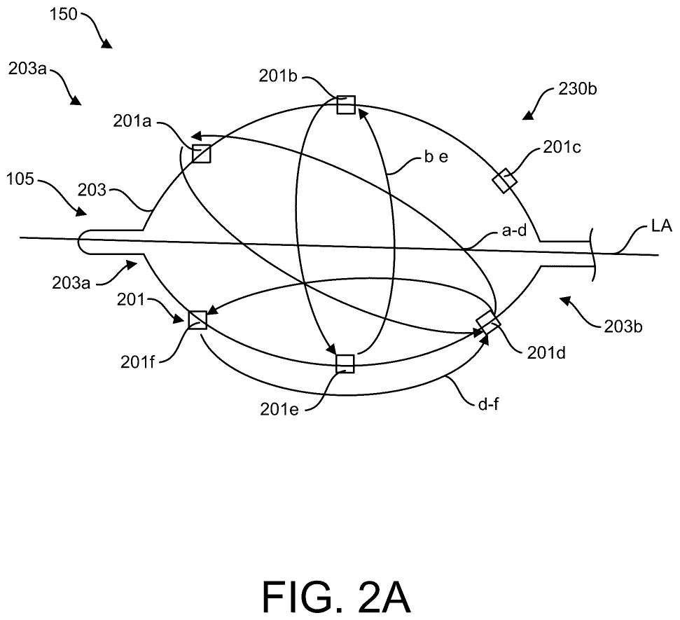

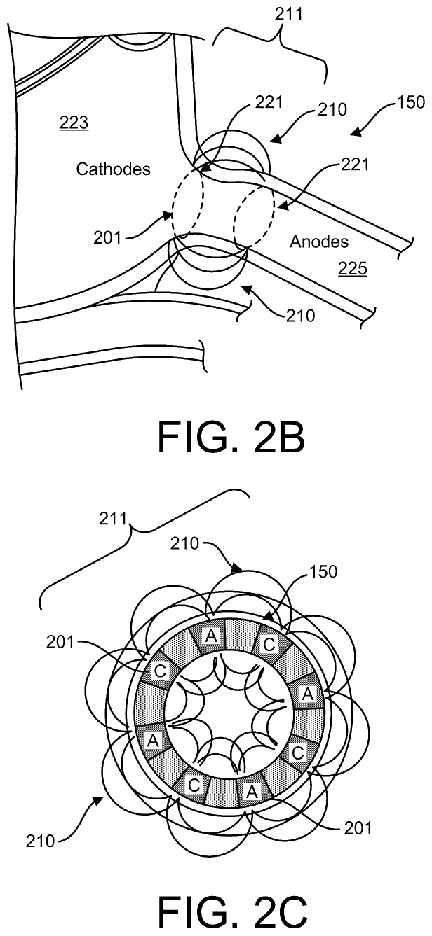

FIGS. 2A-2C show features of the electroporation catheter 105 that includes the electroporation electrode arrangement 150 according to exemplary embodiments. In the illustrated embodiment in FIG. 2A, the electroporation electrode arrangement 150 includes a plurality of electrodes 201a, 201b, 201c, 201d, 201e, and 201f arranged in a three-dimensional electrode array, such that respective ones of the electrodes 201a, 201b, 201c, 201d, 201e, and 201f are spaced from one another axially (i.e., in the direction of the longitudinal axis LA), circumferentially about the longitudinal axis LA and/or radially relative to the longitudinal axis LA. In some embodiments, the electrodes 201a, 201b, 201c, 201d, 201e, and 201f are each individually, selectively addressable via the electroporation generator 130 (FIG. 1) to define a plurality of anode-cathode electrode pairs, each capable of receiving an electrical pulse sequence from the electroporation generator 130 and, consequently, creating an electric field capable of selectively targeting tissue via electroporation, including ablating target tissue via IRE. FIG. 2A schematically illustrates interactions (e.g., current flows forming electric fields) between electrode pairs formed between electrodes 201 (e.g., 201a, 201b, 201c, 201d, 201e, and 201f) included in the electroporation catheter 105. In this figure, interactions are shown as paired arrows (e.g., a-d, b-e, and d-f) indicating current flows between electrodes 201. And electrode pairs (e.g., 201a and 201d, 201b and 201e, and 201d and 201f) are shown with their respective current flows (e.g., a-d, b-e, and d-f) labeled.

FIG. 2B is a diagram illustrating electric fields 210 generated by interactions between electrode pairs in the electroporation catheter 105. In this figure, axially oriented electric fields 210 are shown positioned at an ostium 221 between the left atrium 223 and the left inferior pulmonary vein 225. In embodiments, the axially oriented electric fields 210 are produced by delivering electrical pulses to axially spaced anodes and cathodes.

FIG. 2C is also a diagram illustrating electric fields 210 generated by interactions between electrode pairs in the electroporation catheter 105. But here, the electric fields 210 are circumferentially oriented. In embodiments, the circumferentially oriented electric fields 210 are produced by delivering electrical pulses to circumferentially spaced anodes (“A”) and cathodes (“C”).

FIGS. 2A-2C show that multiple electric fields 210 may be generated simultaneously and/or sequentially and in axial and circumferential orientations. For example, in embodiments, axially and circumferentially oriented electric fields 210 can be generated non-simultaneously in a pre-defined sequence by selectively controlling the timing of the delivery of the electric pulses to the respective electrodes 201. In addition, it is understood that intermittently generated electric fields 210 caused by staggered interactions between sets of electrode pairs and electric field orientations other than axial and circumferential are not beyond the scope of this disclosure.

As may be seen in FIG. 2A, the electroporation electrode arrangement 150 may include a plurality of individually addressable electrodes 201 (e.g., anodes or cathodes) arranged to selectively define a plurality of electrode pairs (e.g., anode-cathode pairs). Each anode-cathode pair may be configured to generate an electric field when a pulse sequence is delivered thereto. The plurality of anode-cathode pairs may include at least two of a first anode-cathode pair, a second anode-cathode pair, and a third anode-cathode pair. The first anode-cathode pair may be arranged to generate a first electric field oriented generally circumferentially relative to the longitudinal axis when a first pulse sequence is delivered thereto. The second anode-cathode pair may be arranged to generate a second electric field oriented generally in a same direction as the longitudinal axis when a second pulse sequence is delivered thereto. The third anode-cathode pair may be arranged to generate a third electric field oriented generally transverse to the longitudinal axis when a third pulse sequence is delivered thereto. In embodiments, any combination of the first, second, and third pulse sequences may be delivered simultaneously or intermittently and may take a variety of forms.

In embodiments, the electroporation electrode arrangement 150 may be configured to structurally arrange the electrodes 201a, 201b, 201c, 201d, 201e, and 201f into a distally-located first region and a more proximally-located second region. As such, electrode pairs may be formed across various electrodes 201 in the electroporation electrode arrangement 150 between first and second regions. For example, the electrodes 201d and 201f may be configured to form an electrode pair. Similarly, the electrodes 201a and 201d or electrodes 201b and 201e or the combination thereof may be selected to form respective electrode pairs. Thus, the electrode pairs may comprise axially spaced electrodes, transversely spaced electrodes, or circumferentially spaced electrodes. Additionally, in embodiments, a given electrode (e.g., 201d) may serve as a common electrode in at least two electrode pairs to generate electric fields 210.

FIG. 2B shows a diagram of exemplary electric fields 210 that may be generated by the electroporation electrode arrangement 150. The electroporation electrode arrangement 150 may be configured to generate a multidirectional electric field 210 when at least one pulse sequence is delivered thereto. The multidirectional electric field 210 may include at least two of the following directions relative to the longitudinal axis: generally axial, circumferential, and transverse. As used herein, transverse may mean at any non-parallel angle relative to the longitudinal axis. As described elsewhere herein, the electroporation electrode arrangement 150 may be configured to operatively couple to an electroporation generator that is configured to generate the at least one pulse sequence. The electroporation electrode arrangement 150 may be configured to receive the at least one pulse sequence from the electroporation generator. Thus, the electroporation electrode arrangement 150 and the electroporation generator may be in operative communication with each other. In this disclosure, such communication may be used to generate electric fields 210 that are at least substantially gapless.

Undesired gaps in electric fields 210 generated by the electroporation electrode arrangement 150 may be limited or at least substantially eliminated. Such gaps may potentially lead to lesion gaps and therefore require multiple repositions of a catheter, for example. Overlapping electric fields 210 may at least substantially limit the number of such gaps. In embodiments, at least some the electric fields 210 generated in the first pulse sequence set may overlap at least partially with each other. For example, adjacent electric fields 210 (e.g., axial, transverse, and/or circumferential) in a combined electric field 211 may intersect one another so that there are limited to no gaps in the combined electric field 211. Overlapping may occur at or near the periphery of adjacent electric fields 210 or may occur over a preponderance or majority of one or more adjacent electric fields 210. In this disclosure, adjacent means neighboring electrodes 201 or electrodes 201 otherwise near each other. The electroporation generator may be configured to generate pulse sequences used in generating overlapping electric fields.

The configuration of the electroporation electrode arrangement 150 in the various embodiments may take on any form, whether now known or later developed, suitable for a three-dimensional electrode structure. In exemplary embodiments, the electroporation electrode arrangement 150 may be in the form of a splined basket catheter, with respective electrodes 201a, 201b, 201c, 201d, 201e, and 201f positioned on a plurality of splines in any manner known in the art. In embodiments, the electroporation electrode arrangement 150 can be formed on an expandable balloon, e.g., with electrodes formed on flexible circuit branches or individual traces disposed on the balloon surface. In other embodiments, the electroporation electrode arrangement 150 may be in the form of an expandable mesh. In short, the particular structure used to form the electroporation electrode arrangement 150 is not critical to the embodiments of the present disclosure.

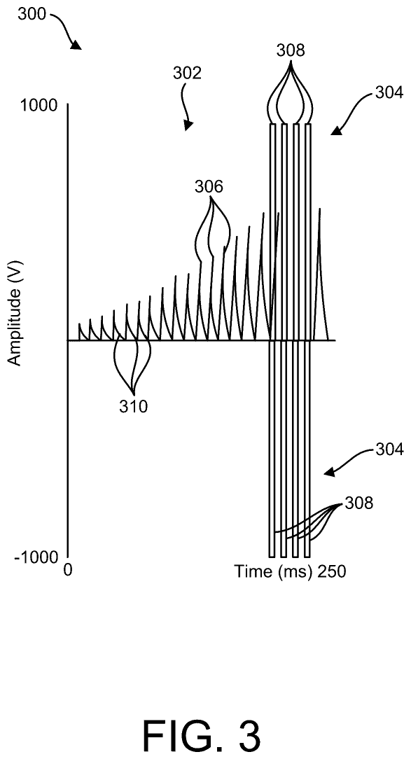

FIG. 3 is a diagram illustrating an ablation energy application sequence 300, also referred to herein as an IRE pulse sequence 300, that includes a preconditioning pulse sequence 302 and an electroporation pulse sequence 304, in accordance with exemplary embodiments. The preconditioning pulse sequence 302 is a series of pulses 306 that ramp up in magnitude from a lower voltage to a higher voltage. The electroporation pulse sequence 304 is a series of high energy electroporation pulses 308 having large positive and/or negative voltage values.

The electroporation system 60 is configured to deliver the preconditioning pulse sequence 302, which delivers the series or sequence of pulses 306 for achieving tetany and for generating electrolysis. In FIG. 3, only three pulses 306 of the preconditioning pulse sequence 302 are pointed to for clarity in the figure, however, in embodiments, the sequence of pulses 306 includes all the pulses in the preconditioning pulse sequence 302. The electroporation system 60 is further configured to deliver the electroporation pulse sequence 304, i.e., electroporation ablation energy, in the series of electroporation pulses 308 to ablate the targeted tissue. In embodiments, all pulses in the IRE pulse sequence 300 are delivered within the refractory period of the heart, such as within 250-330 milliseconds.

The sequence of pulses 306 is a series of monophasic pulses that ramp up in magnitude from a lower voltage to a higher voltage over time. The ramping of voltage causes relatively slower recruitment of skeletal muscles culminating in the tetanic skeletal muscle contraction preceding the higher voltage electroporation pulses 308 in the electroporation pulse sequence 304. Each of the pulses 306, or at least some of the pulses 306, also have features that promote electrolysis, such as including a monophasic exponentially decaying waveform 310 (or drooping waveform) on the back side of the pulse 306. In FIG. 3, only three of the exponentially decaying waveforms 310 in the preconditioning pulse sequence 302 are pointed to for clarity in the figure, however, in embodiments, all the pulses in the preconditioning pulse sequence 302 include exponentially decaying waveforms 310. In embodiments, the pulses 306 may not be completely monophasic, but include at least some asymmetrical phases that are charge balanced with both positive and negative components. In some embodiments, the duration of the pulses 306 is between 1 millisecond (ms) and 60 ms and, in some embodiments, the waveform tilt/droop/exponential decay is in the range of 20 to 80% of the leading edge.

The pulses 306 can have a number of different characteristics. In embodiments, the pulses 306 in the preconditioning pulse sequence 302 are provided at a selected frequency, such as 1 kilohertz. In some embodiments, this frequency is in the range of 200-1000 Hz. In embodiments, the pulses 306 are ramped up in voltage from 0 volts to between 5 and 100 volts and, in some embodiments, the amplitude reaches between 100 and 1000 volts. Also, in embodiments, ramping rates can be incrementing 1% to 30% of the preceding pulse. In some embodiments, the preconditioning pulse sequence 302 is applied in a 100-250 millisecond sequence duration.

The preconditioning pulse sequence 302 preconditions the body for electroporation in at least two ways. First, the preconditioning pulse sequence 302 acts as a series of tetanizing skeletal muscle pulses 306 that cause tetany, i.e., contraction of the skeletal muscles of the patient's body, prior to receiving the higher voltage electroporation pulse sequence 304. Ramping the pulses 306 up in magnitude from a lower voltage to a higher voltage over time causes or contributes to bringing about tetany. This SMS prepares the patient for the higher voltage electroporation pulses 308 in the electroporation pulse sequence 304, which otherwise may shock the patient or be a painful experience for the patient. Second, the preconditioning pulse sequence 302 acts as a series of electrolysis inducing pulses 306 that cause electrolysis near the target tissue. This results in creating a cytotoxic environment near the target tissue, such that smaller electroporation pulses can be used to permeabilize the target tissue and cause cell death, creating larger lesions with smaller electroporation pulses.

This synergistic electrolysis is caused by applying relatively long, low voltage pulses, such as the preconditioning pulse sequence 302, to the target area. In particular, the exponentially decaying waveforms 310 (or drooping waveforms) on the back side of the pulses 306 of the preconditioning pulse sequence 302 bring about electrolysis near the electrodes. In further explanation, synergistic electrolysis occurs when new chemical species are generated at the interface of the electrodes as a result of electron transfer between the electrodes and the ions in solution. The new chemical species diffuse away from the electrodes, into tissue, in a process driven by electrochemical potentials. In physiological solutions, electrolytic reactions yield changes in pH, resulting in an acidic region near the anode and a basic region near the cathode. The cytotoxic environment developing due to local changes in pH, and the presence of some of the new chemical species formed during electrolysis of the solution, along with permeabilization of electroporation cause cell death.

In exemplary embodiments, the preconditioning pulse sequence 302 is delivered as a series of unipolar electrical pulses 306. The surface patch electrode 115, attached to the thorax of the patient 20, receives the electrical pulses from the electroporation generator 130 and sources the electrical energy, where the surface patch electrode 115 acts as a source electrode for the electrical pulses. One or more electrodes on the electroporation catheter 105 sink the electrical energy that is sourced by the surface patch electrode 115, where the one or more electrodes on the electroporation catheter 105 act as a sink for the electrical pulses. This causes the skeletal muscles of the patient to contract, reaching tetany, and generates the synergistic electrolysis near the targeted tissue, where electrodes of the electroporation catheter 105 are situated near the targeted tissue.

In other embodiments, one or more electrodes on the electroporation catheter 105 receives the pulses 306 from the electroporation generator 130 and sources the electrical energy, such that the one or more electrodes on the electroporation catheter 105 act as a source electrode for the electrical pulses 306. The surface patch electrode 115, attached to the thorax of the patient 20, sinks the electrical energy that is sourced by the one or more electrodes on the electroporation catheter 105, such that the surface patch electrode 115 acts as a sink for the electrical pulses.

After delivering at least some of the preconditioning pulse sequence 302, the electroporation system 60 is configured to deliver the electroporation pulse sequence 304, i.e., electroporation ablation energy, to ablate the targeted tissue. The electroporation pulse sequence 304 includes high energy electroporation pulses 308 of short duration. As illustrated, the electroporation pulses 308 of the electroporation pulse sequence 304 are biphasic, including, for example, both positive pulses and negative pulses. In embodiments, the electroporation pulses 308 may be positive 1000 volts and negative 1000 volts. In other embodiments, the electroporation pulses 308 can be monophasic, including, for example, all positive pulses or all negative pulses.

With at least some of the pulses 206 of the preconditioning pulse sequence 302 applied prior to applying the electroporation pulses 308, electrolysis near or at the targeted tissue makes it possible to use electroporation pulses 308 that are lower in amplitude, such as from 250 volts/centimeter (V/cm) to 1000 V/cm, than what is normally or otherwise necessary when using electroporation pulses alone (500 V/cm to 2,500 V/cm).

As illustrated, the electroporation pulses 308 of the electroporation pulse sequence 304 are delivered within the series of pulses 306 of the preconditioning pulse sequence 302. In other embodiments, the electroporation generator 130 can be configured to deliver the electroporation pulses 308 of the electroporation pulse sequence 304 after all the pulses 306 of the preconditioning pulse sequence 302 have been delivered.

The electroporation pulses 308 of the electroporation pulse sequence 304 can be either unipolar pulses or bipolar pulses. In embodiments, pairs of electrodes (or electrode sets) on the electroporation catheter 105 are selected to deliver bipolar pulses between the selected pairs of electrodes. Each electrode of the pairs of electrodes can act as a source electrode and each electrode of the pairs of electrodes can act as a sink, to deliver the electrical ablation energy to the targeted tissue.

In other embodiments, to provide unipolar pulses, one or more electrodes on the electroporation catheter 105 receives the electroporation pulses 308 of the electroporation pulse sequence 304 from the electroporation generator 130 and sources the electrical energy, such that the one or more electrodes on the electroporation catheter 105 act as a source electrode for the electrical pulses. The surface patch electrode 115, attached to the thorax of the patient 20, sinks the electrical energy that is sourced by the one or more electrodes on the electroporation catheter 105, such that the surface patch electrode 115 acts as a sink for the electrical pulses.

In embodiments, the electroporation system 60 includes the accelerometer 117 that is configured to sense contraction of the skeletal muscles of the patient, such as contraction of the patient's chest muscles, to detect tetany. The signals from the accelerometer 117 are received by the electroporation generator 130, which processes the signals to determine whether the skeletal muscle system of the patient is contracting and whether tetany has been achieved. In embodiments, the electroporation generator 130 is configured to provide the electroporation pulse sequence 304 only after tetany has been achieved in the patient. The electroporation generator 130 may provide the electroporation pulse sequence 304 during the preconditioning pulse sequence 302 or after the preconditioning pulse sequence 302.

Thus, the electroporation system 60 acts as a closed system with the surface accelerometer 117 monitoring chest vibrations and the electroporation generator 130 modulating pulses 306 until tetany is achieved and then delivering the electroporation pulses 308. Also, in embodiments, the local impedance of the target tissue and tissue surrounding the target tissue can be measured during this time to calculate pre-ablation and post-ablation values for evaluation of the lesion efficacy.

By applying at least some of the pulses 306 in the preconditioning pulse sequence 302 prior to delivering the electroporation pulses 308 of the electroporation pulse sequence 304, tetany or a contracting of the skeletal muscles of the patient can be achieved and, via electrolysis, a cytotoxic environment can be established near or adjacent the targeted tissue prior to delivering the electroporation pulses 308 of the electroporation pulse sequence 304. This results in being able to use lower energy electroporation pulses 308 and/or fewer electroporation pulses 308 to create the same size lesion as may be created using much more energetic or many more electroporation pulses 308 without the preconditioning pulse sequence 302.

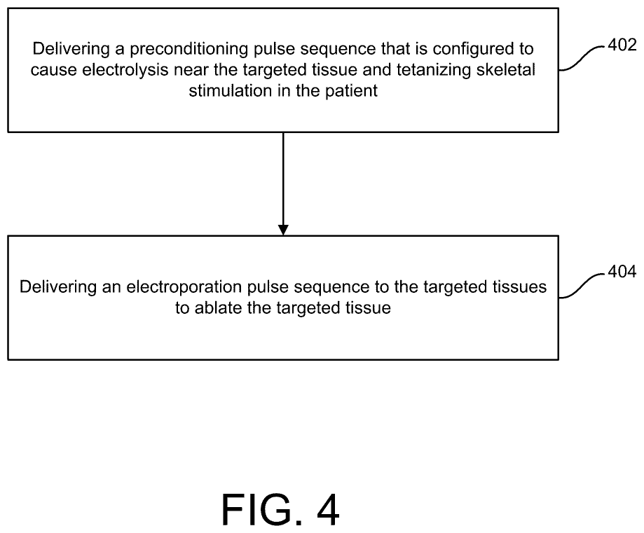

FIG. 4 is a flow chart diagram illustrating a method of ablating targeted tissue in a patient by irreversible electroporation, in accordance with embodiments of the subject matter of the disclosure. Such a method and other related methods of ablating targeted tissue in a patient by irreversible electroporation are disclosed herein.

The method includes delivering an IRE (irreversible electroporation) pulse sequence that includes delivering a preconditioning pulse sequence at 402 and delivering an electroporation pulse sequence at 404. In embodiments, the electroporation pulse sequence is delivered during or within the preconditioning pulse sequence. In other embodiments, the electroporation pulse sequence is delivered after the preconditioning pulse sequence has stopped. In exemplary embodiments, the IRE pulse sequence, including the preconditioning pulse sequence and the electroporation pulse sequence, is delivered to the patient in one or more of a refractory time of the heart of the patient, less than 330 milliseconds, and in a 100-250 millisecond window.

Delivering the preconditioning pulse sequence at 402 includes delivering the preconditioning pulse sequence between a surface patch electrode and one or more catheter electrodes on a catheter to cause electrolysis near the targeted tissue and tetanizing skeletal muscle stimulation in the patient. In embodiments, the preconditioning pulse sequence includes unipolar electrical pulses that are sourced from the surface patch electrode and sunk through the one or more catheter electrodes. In other embodiments, the preconditioning pulse sequence includes unipolar electrical pulses that are sourced from the one or more catheter electrodes and sunk through the surface patch electrode. Also, in embodiments, the preconditioning pulse sequence includes preconditioning pulses that are monophasic.

The preconditioning pulse sequence includes preconditioning pulses delivered at a selected frequency and ramped up in voltage from a lower voltage to a higher voltage over time. In embodiments, the preconditioning pulse sequence includes preconditioning pulses delivered at about 1 kilohertz. Also, in embodiments, the preconditioning pulse sequence includes preconditioning pulses ramped up in voltage from 0 volts to between 5 and 100 volts. Ramping up the voltage of the pulse from a lower voltage to a higher voltage over time, contributes to causing tetanizing skeletal muscle contraction in the patient. Also, the preconditioning pulse sequence includes preconditioning pulses that include an exponentially decaying backside waveform that causes or contributes to electrolysis near the targeted tissue.

Delivering an electroporation pulse sequence at 404 includes delivering the electroporation pulse sequence to catheter electrodes on the catheter to cause irreversible electroporation ablation of the targeted tissue. In embodiments, the electroporation pulse sequence includes bipolar electrical pulses delivered to one or more catheter electrode pairs of the catheter electrodes. In other embodiments, the electroporation pulse sequence includes unipolar electrical pulses delivered between the surface patch electrode and one or more catheter electrodes on the catheter.

In embodiments, the method further includes monitoring the accelerometer 117 on the patient, which is configured to sense contraction of the skeletal muscles of the patient, such as contraction of the patient's chest muscles, to detect tetany. In a closed loop system, the signals from the accelerometer 117 are received by the electroporation generator 130, which processes the signals to determine whether the skeletal muscle system of the patient is contracting and whether tetany has been achieved. In embodiments, the electroporation generator 130 is configured to provide the electroporation pulse sequence only after tetany has been achieved in the patient, which may be during the preconditioning pulse sequence or after the preconditioning pulse sequence.

Also, in embodiments, the method includes monitoring the local impedance of the target tissue and tissue surrounding the target tissue to calculate pre-ablation and post-ablation values for evaluation of the lesion efficacy.

Various modifications and additions may be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

We claim:

1. An apparatus for use in ablating targeted tissue of a patient by irreversible electroporation, the apparatus comprising:
an electroporation generator configured to deliver an irreversible electroporation pulse sequence to one or more catheter electrodes operatively coupled to the electroporation generator,
wherein the irreversible electroporation pulse sequence includes a preconditioning pulse sequence and an electroporation pulse sequence, and
wherein the preconditioning pulse sequence includes preconditioning electrical pulses configured to cause electrolysis near the targeted tissue and tetanizing skeletal muscle stimulation in the patient.

2. The apparatus of claim 1, wherein the preconditioning pulse sequence includes unipolar electrical pulses delivered between the one or more catheter electrodes and a surface patch electrode attached to the body of the patient.

3. The apparatus of claim 1, wherein the preconditioning pulse sequence includes bipolar electrical pulses that are sourced from at least one of the one or more catheter electrodes and sunk through at least another one of the one or more catheter electrodes.

4. The apparatus of claim 3, wherein the preconditioning pulse sequence includes preconditioning pulses delivered at a selected frequency.

5. The apparatus of claim 4, wherein the selected frequency is in the range of 200 Hz-1 KHz.

6. The apparatus of claim 4, wherein the selected frequency is 1000 Hz.

7. The apparatus of claim 6, wherein the preconditioning pulse sequence includes preconditioning pulses ramped up in voltage from a lower voltage to a higher voltage over time.

8. The apparatus of claim 1, wherein the preconditioning pulse sequence includes preconditioning pulses that include an exponentially decaying backside waveform that causes electrolysis near the targeted tissue.

9. The apparatus of claim 1, wherein the preconditioning pulse sequence includes preconditioning pulses that are monophasic.

10. The apparatus of claim 1, wherein the irreversible electroporation pulse sequence, including the preconditioning pulse sequence and the electroporation pulse sequence, is delivered to the patient in one or more of a refractory time of a heart of the patient, less than 330 milliseconds, and in a 100-250 millisecond window.

11. The apparatus of claim 1, wherein the electroporation pulse sequence is delivered within the preconditioning pulse sequence.

12. The apparatus of claim 1, wherein the electroporation pulse sequence includes bipolar electrical pulses delivered to one or more catheter electrode pairs of the catheter electrodes.

13. The apparatus of claim 1, wherein the electroporation generator is configured to receive signals from an accelerometer attached to the body of the patient, and is further configured to process the signals to determine whether tetany has been achieved in response to delivery of the preconditioning pulse sequence.

14. The apparatus of claim 13, wherein the electroporation generator is configured to deliver the electroporation pulse sequency only after tetany has been achieved in the patient.

15. An apparatus for use in ablating targeted tissue of a patient by irreversible electroporation, the apparatus comprising:
an electroporation generator configured to deliver an irreversible electroporation pulse sequence to one or more catheter electrodes operatively coupled to the electroporation generator,
wherein the irreversible electroporation pulse sequence includes a preconditioning pulse sequence and an electroporation pulse sequence,
wherein the preconditioning pulse sequence includes preconditioning electrical pulses configured to cause electrolysis near the targeted tissue and tetanizing skeletal muscle stimulation in the patient, and
wherein the preconditioning pulse sequence includes bipolar electrical pulses that are sourced from at least one of the one or more catheter electrodes and sunk through at least another one of the one or more catheter electrodes.

16. The apparatus of claim 15, wherein the irreversible electroporation pulse sequence, including the preconditioning pulse sequence and the electroporation pulse sequence, is delivered to the patient in one or more of a refractory time of a heart of the patient, less than 330 milliseconds, and in a 100-250 millisecond window.

17. The apparatus of claim 15, wherein the electroporation pulse sequence is delivered within the preconditioning pulse sequence.

18. The apparatus of claim 15, wherein the electroporation pulse sequence includes bipolar electrical pulses delivered to one or more catheter electrode pairs of the catheter electrodes.

19. The apparatus of claim 15, wherein the electroporation generator is configured to receive signals from an accelerometer attached to the body of the patient, and is further configured to process the signals to determine whether tetany has been achieved in response to delivery of the preconditioning pulse sequence.

20. The apparatus of claim 19, wherein the electroporation generator is configured to deliver the electroporation pulse sequency only after tetany has been achieved in the patient.
